(19) United States
(12) Patent Application Publication
(10) Pub. No.: US 2016/0287656 A1
(43) Pub. Date: Oct. 6, 2016

(54) Herbaceutical Formulations

(71) Applicant: Natureceuticals SDN BHD, Bayan Lepas, Pulau Pinang (MY)

(72) Inventors: Amin Malik Shah Bin Abdul Majid, Gelugor, Pulau Pinang (MY); Mahibub Mahamadsa Kanakal, Gombak, Kuala Lumpur (MY)

(21) Appl. No.: 14/778,105

(22) PCT Filed: Mar. 19, 2014
(86) PCT No.: PCT/MY2014/000037

§ 371 Date: Sep. 18, 2015

(30) Foreign Application Priority Data
Mar. 20, 2013 (MY) PI 2013000955

(51) Int. Cl. A61K 36/77; A61K 36/185; A61K 36/19; A61K 36/53

(52) U.S. Cl. CPC A61K 36/77; A61K 36/53; A61K 36/185; A61K 36/19

(57) ABSTRACT

Described are herbaceutical effervescent formulations comprising extracts of Eurycoma longifolia and uses thereof.

### FIELD OF THE INVENTION

The invention described herein relates generally to the field of herbaceutical formulations. In particular, the invention relates to effervescent formulations comprising extracts of Eurycoma longifolia.

### BACKGROUND OF THE INVENTION

Eurycoma longifolia, also known in Malaysia as Tongkat Ali, is a tropical herbal flowering plant in the family Simaroubacae found in several parts of South East Asia, primarily in Malaysia and Indonesia, and, to a lesser extent in Vietnam, Thailand and Laos. Locally it is also known as Payung Ali, Penawar Bias, Setunjang Bumi, Muntah Bumi, Bedara Pahit, Lempedu Pahit, Penawar Pahit, Tongkat Baginda, Pokok Syurga, Tongkat Ali Hitam, Pokok Jelas, Bedara Merah, Bedara Putih and Jelaih. It is referred to as Bidara Laut in Indonesia, cây bá bênh in Vietnamese and tho nan in Laotian.

Extracts of Eurycoma longifolia are generally believed to be useful in the treatment of a wide variety of disorders and syndromes such as: malaria, cancer, anxiety, diabetes, infections, fever, ulcers, male infertility and male sexual dysfunction. Due to the medicinal effects of this plant, extracts of Eurycoma longifolia have high commercial value in both the local and international markets. It has been reported that extracts of Eurycoma longifolia can be sold for US$26.00 per bottle of 60 capsules, which is the equivalent of US$8,700.00/kg extract (Kaur et al., Proceedings of the 17th Symposium of Malaysian Chemical Engineers, 29-30 Dec. 2003, Penang, Malaysia, 294-299).

Extracts of Eurycoma longifolia are, however, extremely bitter making many oral solid formulations difficult to consume. In fact, some of the common names for Eurycoma longifolia refer to the plant's extreme bitterness. “Pahit” in Malay means, “bitter”. “Penawar Pahit”, for example, means, “bitter charm” or “bitter medicine”.

Given the wide variety of therapeutic benefits of Eurycoma longifolia extracts, there is a need for oral formulations of Eurycoma longifolia that mask the extreme bitterness and thus make the extract of this therapeutically valuable herbal medicine, used either alone or in combination with other herbal extracts, therapeutic or health benefiting compounds, easier to consume.

### SUMMARY OF THE INVENTION

In order to facilitate oral administration of a standardized or a non-standardized extract of Eurycoma longifolia, formulations have now been developed to mask the extreme bitterness of the extract to make more palatable to consume.

Accordingly, in at least one embodiment, taste masked herbaceutical formulations comprising an effective amount of a standardized extract of Eurycoma longifolia are provided. In other embodiments, the taste masked formulations comprise an effective amount of a non-standardized extract of Eurycoma longifolia.

In at least one embodiment, the herbaceutical formulation comprises an effective amount of a standardized or non-standardized extract of Eurycoma longifolia, at least one taste masking agent, at least one effervescent couple, least one flavoring agent, at least one lubricant, and at least one mineral salt.

In at least one embodiment, the herbaceutical formulation comprises an effective amount of a bioactive fraction of Eurycoma longifolia, at least one taste masking agent, at least one effervescent couple, at least one flavoring agent, at least one lubricant, and at least one mineral salt.

In at least one embodiment, the bioactive fraction of Eurycoma longifolia comprises a peptide molecule having a molecular weight of 4300 daltons.

In at least one embodiment, the herbaceutical formulation comprises an effective amount of a standardized or non-standardized extract of Eurycoma longifolia; about 0.05% to about 90% by weight of the formulation at least one taste masking agent; about 5% to about 30% by weight of the formulation at least one effervescent couple; about 0.002% to about 30% by weight of the formulation at east one flavoring agent; about 0.05% to about 5% by weight of the formulation at least one lubricant; and, about 0.2% to about 5% by weight of the formulation at least one mineral salt.

In at least one embodiment, the herbaceutical formulation comprises an effective amount of a bioactive fraction of Eurycoma longifolia; about 0.05% to about 90% by weight of the formulation at least one taste masking agent; about 5% to about 30% by weight of the formulation at least one effervescent couple; about 0.002% to about 30% by weight of the formulation at east one flavoring agent; about 0.05% to about 5% by weight of the formulation at least one lubricant; and, about 0.2% to about 5% by weight of the formulation at least one mineral salt.

In at least one embodiment, the herbaceutical formulation comprises an effective amount of a bioactive fraction of Eurycoma longifolia comprising a peptide having a molecular weight of about 4300 daltons; about 0.05% to about 90% by weight of the formulation at least one taste masking agent; about 5% to about 30% by weight of the formulation at least one effervescent couple; about 0.002% to about 30% by weight of the formulation at east one flavoring agent; about 0.05% to about 5% by weight of the formulation at least one lubricant; and, about 0.2% to about 5% by weight of the formulation at least one mineral salt.

In at least one embodiment, the herbaceutical formulation comprises an effective amount of a standardized or non-standardized extract of Eurycoma longifolia from about 0.1% to about 20% by weight of the formulation.

In at least one embodiment, the herbaceutical formulation comprises an effective amount of a standardized or non-standardized extract of Eurycoma longifolia at about 1.1% by weight of the formulation.

In at least one embodiment, the herbaceutical formulation comprises an effective amount of a standardized or non-standardized extract of Eurycoma longifolia and an effective amount of a second standardized or non-standardized extract selected from the group consisting of Orthosiphon stamineus, Labisia pumila, Andrographis paniculata, Phyllanthus niruri, Hibiscus sabdariffa and mixtures thereof.

In at least one embodiment, the herbaceutical formulation comprises an effective amount of a standardized extract of Eurycoma longifolia and a non-standardized extract of Hibiscus sabdariffa.

In at least one embodiment, the herbaceutical formulation comprises an effective amount of a non-standardized extract of Eurycoma longifolia and an effective amount of a non-standardized extract of Hibiscus sabdariffa.

In at least one embodiment, the herbaceutical formulation comprises about 1.1% by weight of the formulation a non-standardized extract of Eurycoma longifolia and about 5.2% of a non-standardized extract of Hibiscus sabdariffa.

In at least one embodiment, the non-standardized extract of Eurycoma longifolia comprises a water-soluble extract of Eurycoma longifolia.

In at least one embodiment the second non-standardized extract of an herbal plant comprises a water-soluble extract of said herbal plant. Accordingly, in at least one embodiment, the non-standardized extract of Hibiscus sabdariffa comprises a water-soluble extract of Hibiscus sabdariffa.

In at least one embodiment, the herbaceutical formulation comprises an effective amount of a standardized or non-standardized extract of Eurycoma longifolia and an effective amount of a compound selected from the group consisting of chondroitin sulfate, glucosamine, methylsulfonylmethane, bromelain and mixtures thereof.

In at least one embodiment, the herbaceutical formulation comprises an effective amount of a standardized or non-standardized extract of Eurycoma longifolia and an effective amount of glucosamine.

In at least one embodiment, the herbaceutical formulation comprises an effective amount of a standardized or non-standardized extract of Eurycoma longifolia and an effective amount of chondroitin sulfate.

In at least one embodiment, the herbaceutical formulation comprises an effective amount of a standardized or non-standardized extract of Eurycoma longifolia and an effective amount of a mixture of glucosamine and chondroitin sulfate.

In at least one embodiment, the herbaceutical formulation comprises at least one taste masking agent selected from the group consisting of monosaccharides; disaccharides; sugar alcohols; polydextrose; dextrates; maltodextrin; sugar substitutes; cyclamate; β-cyclodextrin, Dimethyl-β-cyclodextrin; 2-hydroxyethyl-β-cyclodextrin; 2-hydroxypropyl-β-cyclodextrin; 3-hydroxypropyl-β-cyclodextrin; trimethyl-β-cylcodextrin and mixtures thereof.

In at least one embodiment, the herbaceutical formulation comprises at least one taste masking agent selected from the group consisting of monosaccharides; sugar substitutes; β-cyclodextrin and mixtures thereof.

In at least one embodiment, the herbaceutical formulation comprises about 5% to about 99% by weight of the formulation a first taste masking agent selected from the group consisting of monosaccharides, disaccharides, sugar alcohols, polydextrose, dextrates, maltodextrin and mixtures thereof; about 0.05% to about 5% by weight of the formulation a second taste masking agent selected from the group consisting of sugar substitutes, cyclamate and mixtures thereof; about 0.2% to about 40% by weight of the formulation a third taste masking formulation selected from the group consisting of β-cyclodextrin, Dimethyl-β-cyclodextrin; 2-hydroxyethyl-β-cyclodextrin; 2-hydroxypropyl-β-cyclodextrin; 3-hydroxypropyl-β-cyclodextrin; trimethyl-β-cylcodextrin and mixtures thereof.

In at least one embodiment, the herbaceutical formulation comprises about 20% to about 90% by weight of the formulation a first taste masking agent selected from the group consisting of monosaccharides, disaccharides, sugar alcohols, polydextrose, dextrates, maltodextrin and mixtures thereof; about 0.1% to about 3% by weight of the formulation a second taste masking agent selected from the group consisting of sugar substitutes, cyclamate and mixtures thereof; about 0.4% to about 10% by weight of the formulation a third taste masking formulation selected from the group consisting of β-cyclodextrin, Dimethyl-β-cyclodextrin; 2-hydroxyethyl-β-cyclodextrin; 2-hydroxypropyl-β-cyclodextrin; 3-hydroxypropyl-β-cyclodextrin; trimethyl-β-cylcodextrin and mixtures thereof.

In at least one embodiment, the herbaceutical formulation comprises about 30% to about 70% by weight of the formulation a first taste masking agent selected from the group consisting of monosaccharides, disaccharides, sugar alcohols, polydextrose, dextrates, maltodextrin and mixtures thereof; about 0.5% to about 2% by weight of the formulation a second taste masking agent selected from the group consisting of sugar substitutes, cyclamate and mixtures thereof; about 1% to about 5% by weight of the formulation a third taste masking formulation selected from the group consisting of β-cyclodextrin, Dimethyl-β-cyclodextrin; 2-hydroxyethyl-β-cyclodextrin; 2-hydroxypropyl-β-cyclodextrin; 3-hydroxypropyl-3-cyclodextrin; trimethyl-β-cylcodextrin and mixtures thereof.

In at least one embodiment of the herbaceutical formulation, the at least one taste masking agent comprises glucose; 1,6-Dichloro-1,6-dideoxy-β-D-fructofuranosyl-4-chloro-4-deoxy-α-D-galactopyranoside and β-cyclodextrin.

In at least one embodiment, the herbaceutical formulation comprises by weight of the formulation about 5% to about 99% glucose, about 0.05% to about 5% 1,6-Dichloro-1,6-dideoxy-β-D-fructofuranosyl-4-chloro-4-deoxy-α-D-galactopyranoside and about 0.2% to about 40% β-cyclodextrin.

In at least one embodiment, the herbaceutical formulation comprises by weight of the formulation about 20% to about 90% glucose, about 0.1% to about 3% 1,6-Dichloro-1,6-dideoxy-β-D-fructofuranosyl-4-chloro-4-deoxy-α-D-galactopyranoside and about 0.4% to about 10% β-cyclodextrin.

In at least one embodiment, the herbaceutical formulation comprises by weight of the formulation about 30% to about 70% glucose, about 0.5% to about 2% 1,6-Dichloro-1,6-dideoxy-β-D-fructofuranosyl-4-chloro-4-deoxy-α-D-galactopyranoside and about 1% to about 5% β-cyclodextrin.

In at least one embodiment of the herbaceutical formulation, the at least one taste masking agent comprises by weight of the formulation about 66% glucose, about 1.7% 1,6-Dichloro-1,6-dideoxy-β-D-fructofuranosyl-4-chloro-4-deoxy-α-D-galactopyranoside and about 1.1%β-cyclodextrin.

In at least one other embodiment of the herbaceutical formulation, the at least one taste masking agent comprises by weight of the formulation about 63% glucose, about 1.6% 1,6-Dichloro-1,6-dideoxy-β-D-fructofuranosyl-4-chloro-4-deoxy-α-D-galactopyranoside and about 1% β-cyclodextrin.

In at least one embodiment, the herbaceutical formulation comprises at least one flavoring agent selected from the group consisting of citron, menthol, mint, thymol, a flavoring agent derived from fruit, anhydrous citric acid, tartaric acid, malic acid, sodium gluconate, trisodium citrate, fumaric acid, adipic acid and mixtures thereof.

In at least one embodiment, the herbaceutical formulation comprises by weight of the formulation about 0.002% to about 30% by weight of the formulation at least one flavoring agent selected from the group consisting of citric oils, such as lemon, citron, orange, grape, lime and grapefruit, and fruit essences, including apple, pear, peach, grape, strawberry, raspberry, cherry, plum, pineapple, apricot, or other fruit flavors, aldehydes and esters, such as benzaldehyde (cherry, almond); citral, i.e., alpha-citral (lemon, lime); neral, i.e., beta-citral (lemon, lime); decanal (orange, lemon); aldehyde C-8 (citrus fruits); aldehyde C-9 (citrus fruits); aldehyde C-12 (citrus fruits); tolyl aldehyde (cherry, almond); 2,6-dimethyloctanal (green fruit); 2-dodenal (citrus mandarin); and mixtures thereof.

In at least one embodiment, the herbaceutical formulation comprises by weight of the formulation about 0.1% to about 20% by weight of the formulation at least one flavoring agent selected from the group consisting of citric oils, such as lemon, citron, orange, grape, lime and grapefruit, and fruit essences, including apple, pear, peach, grape, strawberry, raspberry, cherry, plum, pineapple, apricot, or other fruit flavors, aldehydes and esters, such as benzaldehyde (cherry, almond); citral, i.e., alpha-citral (lemon, lime); neral, i.e., beta-citral (lemon, lime); decanal (orange, lemon); aldehyde C-8 (citrus fruits); aldehyde C-9 (citrus fruits); aldehyde C-12 (citrus fruits); tolyl aldehyde (cherry, almond); 2,6-dimethyloctanal (green fruit); 2-dodenal (citrus mandarin); and mixtures thereof.

In at least one embodiment, the herbaceutical formulation comprises about 0.5% to about 10% by weight of the formulation at least one flavoring agent selected from the group consisting of citric oils, such as lemon, citron, orange, grape, lime and grapefruit, and fruit essences, including apple, pear, peach, grape, strawberry, raspberry, cherry, plum, pineapple, apricot, or other fruit flavors, aldehydes and esters, such as benzaldehyde (cherry, almond); citral, i.e., alpha-citral (lemon, lime); neral, i.e., beta-citral (lemon, lime); decanal (orange, lemon); aldehyde C-8 (citrus fruits); aldehyde C-9 (citrus fruits); aldehyde C-12 (citrus fruits); tolyl aldehyde (cherry, almond); 2,6-dimethyloctanal (green fruit); 2-dodenal (citrus mandarin); and mixtures thereof.

In at least one embodiment the herbaceutical formulation comprises about 7.5% to about 30% by weight of the formulation a first flavoring agent selected from the group consisting of anhydrous citric acid, tartaric acid, sodium gluconate, trisodium citrate, fumaric acid, malic acid, adipic acid and mixtures thereof; about 0.1% to about 9% by weight of the formulation a second flavoring agent selected from the group consisting of a fruit based flavoring agent; and about 0.002% to about 4% by weight of the formulation a third flavoring agent selected from the group consisting of menthol, mint, thymol and mixtures thereof.

In at least one embodiment the herbaceutical formulation comprises about 10% to about 20% by weight of the formulation a first flavoring agent selected from the group consisting of anhydrous citric acid, tartaric acid, sodium gluconate, trisodium citrate, fumaric acid, malic acid, adipic acid and mixtures thereof; about 0.5% to about 4% by weight of the formulation a second flavoring agent selected from the group consisting of a fruit based flavoring agent; and about 0.1% to about 1% by weight of the formulation a third flavoring agent selected from the group consisting of menthol, mint, thymol and mixtures thereof.

In at least one embodiment of the herbaceutical formulation, the flavoring agent comprises about 0.1% to about 9% citron and about 0.05% to about 4% menthol by weight of the formulation.

In at least one embodiment of the herbaceutical formulation, the at least one flavoring agent comprises about 2.2% citron and about 0.14% menthol by weight of the formulation.

In at least one other embodiment of the herbaceutical formulation, the at least one flavoring agent comprises about 2% citron and about 0.14% menthol by weight of the formulation.

In at least one embodiment, the herbaceutical formulation comprises an effervescent couple comprising least one acidic component comprising about 7.5% to about 30% by weight of the formulation wherein the acidic component is selected from the group consisting of anhydrous citric acid, tartaric acid, sodium gluconate, trisodium citrate, fumaric acid, malic acid, adipic acid or a combination of any acid salts thereof; and at least one alkaline component comprising about 5% to about 25% by weight of the formulation wherein the alkaline component is selected from the group consisting of anhydrous sodium, potassium, calcium (bi) carbonates, sodium glycine carbonates and mixtures thereof.

In at least one embodiment, the herbaceutical formulation comprises an effervescent couple comprising at least one acidic component comprising about 10% to about 20% by weight of the formulation wherein the acidic component is selected from the group consisting of anhydrous citric acid, tartaric acid, sodium gluconate, trisodium citrate, fumaric acid, malic acid, adipic acid or a combination of any acid salts thereof; and at least one alkaline component comprising about 10% to about 15% by weight of the formulation wherein the alkaline component is selected from the group consisting of anhydrous sodium, potassium, calcium (bi) carbonates, sodium glycine carbonates and mixtures thereof.

In at least one embodiment of the herbaceutical formulation, the at least one effervescent couple comprises about 13% by weight of the formulation anhydrous citric acid and about 11% by weight of the formulation anhydrous sodium bicarbonate.

In at least one other embodiment of the herbaceutical formulation, the at least one effervescent couple comprises about 12.6% anhydrous citric acid and about 10.5% anhydrous sodium bicarbonate.

In at least one embodiment, the herbaceutical formulation comprises at least one lubricant selected from the group consisting of magnesium aluminum silicate, hydroxyethyl cellulose, lauric acid, leucine, poloxamers, polyvinyl alcohol, talc, calcium stearate, glyceryl monostearate, glyceryl behenate, glyceryl palmitostearate, magnesium lauryl sulfate, sodium lauryl sulfate, sodium stearyl fumarate, stearic acid, magnesium stearate, medium chain triglycerides, palmitic acid, polyethylene glycol, potassium benzoate, sodium benzoate, zinc stearate and mixtures thereof.

In at least one embodiment, the herbaceutical formulation comprises about 0.05% to about 5% by weight of the formulation at least one lubricant selected from the group consisting of magnesium aluminum silicate, hydroxyethyl cellulose, lauric acid, leucine, poloxamers, polyvinyl alcohol, talc, calcium stearate, glyceryl monostearate, glyceryl behenate, glyceryl palmitostearate, magnesium lauryl sulfate, sodium lauryl sulfate, sodium stearyl fumarate, stearic acid, magnesium stearate, medium chain triglycerides, palmitic acid, polyethylene glycol, potassium benzoate, sodium benzoate, zinc stearate and mixtures thereof.

In at least one embodiment, the herbaceutical formulation comprises about 0.1% to about 2% by weight of the formulation at least one lubricant selected from the group consisting of magnesium aluminum silicate, hydroxyethyl cellulose, lauric acid, leucine, poloxamers, polyvinyl alcohol, talc, calcium stearate, glyceryl monostearate, glyceryl behenate, glyceryl palmitostearate, magnesium lauryl sulfate, sodium lauryl sulfate, sodium stearyl fumarate, stearic acid, magnesium stearate, medium chain triglycerides, palmitic acid, polyethylene glycol, potassium benzoate, sodium benzoate, zinc stearate and mixtures thereof.

In at least one embodiment, the herbaceutical formulation comprises about 0.4% by weight of the formulation magnesium aluminum silicate.

In at least one other embodiment, the herbaceutical formulation comprises about 7% by weight of the formulation magnesium aluminum silicate.

In at least one embodiment, the herbaceutical formulation comprises about 0.2% to about 5% by weight of the formulation at least one mineral salt selected from the group consisting of potassium chloride, sodium chloride, magnesium chloride, calcium chloride and mixtures thereof.

In at least one embodiment, the herbaceutical formulation comprises about 0.8% to about 5% by weight of the formulation potassium chloride and about 0.2% to about 1.25% by weight of the formulation sodium chloride.

In at least one embodiment, the herbaceutical formulation comprises about 2.2% by weight of the formulation potassium chloride and about 0.6% by weight of the formulation sodium chloride.

In at least one other embodiment, the herbaceutical formulation comprises about 2% by weight of the formulation potassium chloride and about 0.5% by weight of the formulation sodium chloride.

In at least one embodiment, the herbaceutical formulation is used as a sports drink.

In at least one embodiment, the herbaceutical formulation is used to enhance athletic performance in a human subject, said human subject being either a male or a female.

In at least one embodiment, the herbaceutical formulation is used for treating male infertility, male sexual dysfunction, increasing testosterone synthesis, increasing testosterone release from testis cells, increasing sperm count, increasing sperm motility, treating the symptoms of late-onset hypoganadism and the management of hypogonadism in a human male in need of such treatment.

In at least one embodiment, the herbaceutical formulation is used to enhance sexual libido in a human subject, said subject being either a male or female.

In at least one embodiment, the herbaceutical formulation is manufactured as an effervescent powder for oral administration. In at least one other embodiment, the effervescent powder is compressed into a tablet.

In at least one embodiment, the herbaceutical formulation is manufactured as a divided powder, wherein the divided powders are separately packaged into one or more sachets.

In at least one embodiment, the herbaceutical formulation is manufactured as effervescent granules for oral administration. In at least one other embodiment, the effervescent granules are compressed into a tablet.

In at least one embodiment, the herbaceutical formulation is manufactured as divided granules, wherein one or more of the divided granules are separately packaged into one or more sachets.

In at least one embodiment, the herbaceutical formulation is manufactured as an effervescent oral powder or tablet and is packaged into a unit-dosage form in a sachet.

In at least one embodiment, a method is provided for enhancing athletic performance in a human subject wishing to enhance his athletic performance, which method comprises administering to the subject an effective amount of the effervescent herbal formulation comprising an effective amount of a standardized or non-standardized extract of Eurycoma longifolia described herein. In at least one embodiment, said human subject is a male. In at least one other embodiment, said human subject is a female.

In at least one embodiment, the effervescent herbal formulations described herein can be manufactured as powders and packaged as sachets with instructions for using the formulations. For example, the instructions can be provided as a package insert or directly on a label attached to the sachets, and/or on secondary packaging in which the sachets are finally packaged in for shipment to distributors or retailers. The instructions can include, for example, dosing frequency, method of dissolving the powdered formulation in an aqueous medium for consumption, the active ingredients comprising the composition, and the conditions that would benefit from administration of the formulations.

### BRIEF DESCRIPTION OF THE DRAWINGS

FIG. 1 shows a schematic flowchart of the process for manufacturing at least one embodiment of the herbaceutical formulation described herein.

### DETAILED DESCRIPTION

### Definitions

As used herein, “standardized extract” means a form of an extract derived form of an herb comprising a concentrated but set (standardized) percentage of medicinally active botanical ingredients. The standardized extract may be in the form of a liquid, solid or emulsion. Standardized extracts provide consistent therapeutic dosage strength, or therapeutic potency, from one batch of the herb to the next. Methods of standardizing herbal extracts are well known to the person of ordinary skill in the art. For example, one method of standardizing an herbal extract is to identify and quantify the extract to one or more known chemical marker compounds. Another method identifies and concentrates one or more bioactive phytoconstituents in a defined and optimum proportion in the extract. Phytoconstituents are chemical compounds present in the standardized extract that account for the medicinal properties of the standardized extract. Standardized extracts comprising such phytoconstituents exhibit therapeutic efficacy. Bioactive compounds present in standardized extracts may comprise proteins, polysaccharides, glycosaponins, phenolic compounds, flavonoids or mixtures thereof. Methods for standardizing herbal extracts include the use of analytical instruments such as HPLC, Mass Spectrometry (MS), Nuclear Magnetic Resonance (NMR) and Thin Layer Chromatography (TLC). Standardized extracts can be manufactured based on standard solvent extraction, where the solvent can be aqueous or organic, or supercritical fluid extraction employing liquid CO₂ as a solvent system. One common organic solvent used is alcohol. One common aqueous solvent used is water. Accordingly, standardized extracts can be water or alcohol based. Standardized liquid extracts may be further spray dried to form a powder.

“Non-standardized extract” means a form of an extract derived from an herb wherein the active component(s) may not have been completely identified and the percentages of these active component(s) have not been quantified. Non-standardized extracts may be obtained using an organic or aqueous solvent. Typically, the solvent is water. Non-standardized liquid extracts may be further spray dried to form a powder. While physicians may not prescribe formulations comprising non-standardized extracts of herbal plants to treat a certain pathological condition, such extracts may nevertheless provide health benefits. For example, non-standardized extracts, in addition to the bioactive(s) may also be rich in vitamins, minerals, flavonoids, anti-oxidants or mixtures thereof.

The term “effective amount” or an “amount effective” is an amount shown to provide a beneficial effect or response when administered to a human subject.

The term “powder” is used to describe a dosage form in which the herbaceutical extract in powder form is mixed with other powdered excipients to produce the final product, which can be packaged directly into sachets. The powder can also be used to further form granules, which can then be packaged into sachets. The powder or granules can also be compressed into tablets, which tablets can then be packaged into sachets. The formulation can comprise one homogenous mixture of the powder or can comprise a divided powder. For example, in a divided powder one or more components of the formulation can be packaged into separate sachets. The formulation can be reconstituted by combining the powders of two or more sachets prior to consumption.

The term “granules” comprise aggregated powder particles to form larger particles. Such granules can be directly packaged into sachets or compressed into tablets that can also be packaged into sachets. Like a powder, a formulation comprising a granule can comprise one homogenous mixture of granules or can comprise a divided granular composition. For example, in a divided granular composition, one or more components of the formulation can be packaged into separate sachets. The formulation can be reconstituted by combining the granules of two or more sachets prior to consumption.

The term “about” or “approximately” as used herein means within an acceptable error range for the particular value as determined by one of ordinary skill in the art, which will depend in part on how the value is measured or determined, i.e., the limitations of the measurement system. Where particular values are described in the application and claims, unless otherwise stated, the term “about” means within an acceptable error range for the particular value.

Herbaceutical Formulations

The present invention is directed to taste masked herbaceutical formulations comprising an effective amount of a standardized extract of Eurycoma longifolia. The present invention is also directed to taste masked herbaceutical formulations comprising an effective amount of a non-standardized extract of Eurycoma longifolia.

In at least one embodiment, the herbaceutical formulation comprises an effective amount of a standardized extract of Eurycoma longifolia, at least one taste masking agent, at least one effervescent couple; least one flavoring agent, at least one lubricant, and at least one mineral salt.

In at least one embodiment, the herbaceutical formulation comprises an effective amount of a non-standardized extract of Eurycoma longifolia, at least one taste masking agent, at least one effervescent couple; least one flavoring agent, at least one lubricant, and at least one mineral salt.

The standardized extract of Eurycoma longifolia used herein was obtained from Biotropics Malaysia Berhad (Biotropics Malaysia Berhad, Shah Alam, Selangor Darul Ehsan, Malaysia) and is described in U.S. Pat. No. 7,132,117 (the '117 patent). The standardized extract of Eurycoma longifolia comprises a peptide molecule having a molecular weight of about 4300 daltons as the bioactive component. The '117 patent describes the extract as being useful for the treatment of male infertility and sexual dysfunction. Male infertility encompasses many aspects including but not limited to increasing testosterone synthesis, increasing testosterone release from testis cells, increasing sperm count, or increasing sperm motility. Standardized water soluble extracts of Eurycoma longifolia have also been reported to treat symptoms of late-onset hypogonadism and management of hypogonadism (Tambi et. Al. Andrologia. 2012 May; 44 Suppl 1:226-30. doi: 10.1111/j.1439-0272.2011.01168.x. Epub 2011 June 15). Accordingly, the herbaceutical formulation described herein may be used for the treatment of male infertility or sexual dysfunction including but not limited to increasing testosterone synthesis, increasing testosterone release from testis cells, increasing sperm count, increasing sperm motility, treating symptoms of late-stage hypogonadism or management of hypogonadism.

Non-standardized extracts of Eurycoma longifolia used herein was obtained from the Institute of Bioproduct Development, Universiti Teknologi Malaysia, Johor, Malaysia. Typically, the non-standardized extracts are water-soluble extracts.

The standardized or non-standardized extracts of Eurycoma longifolia also comprise quassinoids such as eurycomalacton, eurycomanon, and eurycomanol, which have been reported to have aphrodisiac properties and to increase testosterone levels in men. Previous studies have established that the testosterone supplementation increases fat free mass, muscle strength, and muscle mass, which are important for physical function and athletic performance (Hamzah, et. al. Br J Sports Med 2003; 37:464-470). Accordingly, in one embodiment, the herbaceutical formulation comprising a standardized or non-standardized extract of Eurycoma longifolia described herein can also be used as a sports drink or tonic.

The amount of the standardized extract of Eurycoma longifolia, will depend on the condition to be treated or the purpose for which it is to be used. In at least one embodiment, the amount of the standardized or non-standardized extract used in the taste masked herbal formulation comprises about 0.1, 0.25, 0.5, 1, 1.25, 1.5, 1.75, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5 or 20% by weight of the formulation. In a preferred embodiment, the amount of the standardized or non-standardized extract used in the taste masked herbal formulation can be about 1.25% by weight of the desired formulation.

The standardized or non-standardized extract of Eurycoma longifolia can also be used in combination with one or more standardized or non-standardized extracts of other herbal plants. Examples of other standardized or non-standardized herbal extracts include, but are not limited to, extracts of Orthosiphon stamineus, Labisia pumila, Andrographis paniculata, Phyllanthus niruri, and Hibiscus sabdariffa. Some of these other standardized or non-standardized herbal extracts can also function as flavourants in addition to having therapeutic properties or health benefits. An example of such an herbal extract is one derived from Hibiscus sabdariffa. For example, non-standardized water-soluble extracts of Hibiscus sabdariffa have been shown to have anti-oxidant and anti-proliferative properties in-vitro (Akim et. al. 2011. Antioxidant and anti-proliferative activities of Roselle juice on Caov-3, MCF-7, MDA-MB-231 and HeLa cancer cell lines. African Journal of Pharmacy and Pharmacology Vol. 5(7), pp. 957-965). In addition, the extract can also be used to impart a deep red color and acidic rhubarb-like flavor to the formulation.

Standardized or non-standardized herbal extracts from other herbal plants can also be included in combination with an effective amount of standardized or non-standardized extracts of Eurycoma longifolia for other health benefits. For example, water-soluble extracts of Hibiscus sabdariffa are also known to be rich in vitamins B1, B2, B3 and C.

The extract of Eurycoma longifolia, whether standardized or not, can also be used in combination with other health benefiting compounds. Examples of such compounds include, but are not limited to, flavonoids, anti-oxidants and nutritional supplements such as glucosamine, chondroitin sulfate, methylsulfonylmethane and bromelain.

It will be appreciated that the precise therapeutic dose of a standardized extract of Eurycoma longifolia, used alone or in combination with a standardized extract from another herbal plant or in combination with another therapeutic compound, will depend on the age and condition of the patient as well as the nature of the condition to be treated and will be at the ultimate discretion of the attendant physician.

The standardized or non-standardized extract of Eurycoma longifolia, as well as the standardized or non-standardized extracts of other herbal plants, is preferably in solid form such as in a powder or granular form.

Taste masking agents are especially suitable for improving the taste characteristics of a wide variety of medicaments, particularly for improving the taste of bitter tasting medicaments. The class of taste masking agents suitable for use in formulations comprising standardized or non-standardized extracts of Eurycoma longifolia include for example, the use of sweeteners such as monosaccharides, disaccharides, sugar alcohols, polydextrose, dextrates, dextrin, dextrose anhydrous, sugar substitutes and mixtures thereof.

In at least one embodiment, the amount of sweetener comprises about 5, 10, 15, 20, 25, 30, 35, 40, 45, 50, 55, 60, 65, 70, 75, 80, 85, 90 or 99% by weight of the formulation depending on the amount of the standardized or non-standardized extract of Eurycoma longifolia used, as well as the amount of the other excipients used to manufacture the desired formulation. In a preferred embodiment, the amount of sweetener used can be about 68% by weight of the desired formulation.

Examples of monosaccharides suitable for manufacture of the herbaceutical formulations described herein include, but are not limited to, glucose (dextrose), fructose (levulose), galactose, xylose and ribose.

Maltose, lactose, and sucrose are examples of disaccharides that can be used in some embodiments of the herbaceutical formulations described herein as taste masking agents.

Dextrins are low-molecular weight carbohydrates formed by the hydrolysis of starch or glycogen. Examples of dextrins include maltodextrin and the cyclodextrins. Cyclodextrins are of three types: α-cyclodextrin, β-cyclodextrin, and γ-cyclodextrin. β-cyclodextrin is the most accessible and generally the most useful as a taste masking agent in the herbaceutical formulations described herein. β-cyclodextrin can be substituted with dimethyl-β-cyclodextrin; 2-hydroxyethyl-β-cyclodextrin; 2-hydroxypropyl-β-cyclodextrin; β-hydroxypropyl-β-cyclodextrin; trimethyl-β-cylcodextrin or combinations thereof.

Dextrose anhydrous, another sweetener that can be used in at least one embodiment as a taste masking agent, is manufactured from refining dextrose monohydrate by removing its one water molecule.

In some embodiments, polydextrose, an indigestible synthetic polymer of glucose, can also be used as a taste masking agent.

Examples of sugar alcohols that can be used in the herbaceutical formulations described herein include, but are not limited to, erythritol, hydrogenated starch hydrolysates, isomalt, lactitol, maltitol, mannitol, sorbitol and xylitol.

Certain embodiments of the herbaceutical formulation described herein use Generally Accepted As Safe (GRAS) artificial sweeteners or sugar substitutes. Examples of such artificial sweeteners include, but are not limited to, acesulfame potassium (potassium 6-methyl-2,2-dioxo-2H-1,2λ⁶,3-oxathiazin-4-olate), aspartame (N-(L-α-Aspartyl)-L-phenylalanine, [symbol] 1-methyl ester), neotame ((3S)-3-(3,3-Dimethylbutylamino)-4-[[(2S)-1-methoxy-1-oxo-3-phenylpropan-2-yl]amino]-4-oxobutanoic acid), saccharin (2H-1λ⁶,2-benzothiazol-1,1,3-trione), sucralose (1,6-Dichloro-1,6-dideoxy-β-D-fructofuranosyl-4-chloro-4-deoxy-α-D-galactopyranoside), glycyrrhizin ((3β,18α)-30-hydroxy-11,30-dioxoolean-12-en-3-yl 2-O-β-D-glucopyranuronosyl-β-D-glucopyranosiduronic acid), tagatose ((3S,4S,5R)-1,3,4,5,6-Pentahydroxy-hexan-2-one), or steviol glycosides such as stevioside, rebaudioside A, rebaudioside C, or dulcoside A, alitame ((3S)-3-amino-4-[[(1R)-1-methyl-2-oxo-2-[(2,2,4,4-tetramethyl-3-thietanyl)amino]ethyl]amino]-4-oxobutanoic acid), or cyclamate (sodium or calcium salt). It will be appreciated that not all sugar substitutes have been approved for use in all countries of the world. Accordingly, it will be necessary to determine which sugar substitutes are approved in the country in which the formulation is to be commercialized and the herbaceutical formulation manufactured accordingly.

Combinations of any of the taste masking agents can be used. In at least one embodiment, the combination of taste masking agent used comprises a monosaccharide such as glucose, an artificial sweetener such as saralose and a β-cyclodextrin. In one embodiment, the amount of monosaccharide can comprise about 5, 10, 15, 20, 25, 30, 35, 40, 45, 50, 55, 60, 65, 70, 75, 80, 85, 90 or 99% of the weight of the formulation, preferably about 20, 25, 30, 35, 40, 45, 50, 55, 60, 65, 70, 75, 80, 85 or 90% by weight of the formulation, the artificial sweetener can comprise about 0.05, 0.1, 0.2, 0.4, 0.8, 1, 1.5, 2, 2.5, 3, 3.5, 4, 4.5 or 5% by weight of the formulation, preferably about 0.1, 0.2, 0.4, 0.6, 0.8, 1.0, 1.5, 2, 2.5 or 3% by weight of the formulation, and the 3-cyclodextrin can comprise about 0.2, 0.4, 0.6, 0.8, 1, 2, 4, 6, 8, 10, 15, 20, 25, 30, 35 or 40% by weight of the formulation, preferably about 0.4, 0.6, 0.8, 1, 2, 4, 6, 8 or 10% by weight of the formulation. In one embodiment, the combination of taste masking agents can comprise about 60, 62, 64, 66, 68 or 70% by weight of the formulation. In a preferred embodiment, the combination of taste masking agents comprises about 67.5% by weight of the formulation. In at least one such embodiment, the preferred combination of taste masking agents comprises glucose, sucralose and β-cyclodextrin in about 63%, 2% and 2.5% by weight of the formulation respectively.

The inclusion of an effervescent couple comprising at least one acidic component and at least one base component can improve the palatability of bitter tasting medicaments. The basic component liberates carbon dioxide when it and the acidic component are contacted with an aqueous medium such as water, juice or other beverage. The ‘fizzy’ mouth-feel generated by the effervescence couple is well accepted by patients who would otherwise find the bitter taste of the standardized or non-standardized extract of Eurycoma longifolia difficult to take.

Accordingly, at least one embodiment of the herbaceutical formulation comprises an effervescent couple comprising at least one acidic component and at least one basic component. The effervescent couple typically comprises citric acid or any acid salts thereof, such as trisodium citrate, and sodium bicarbonate, but other physiologically acceptable acid/alkaline or alkaline earth metal carbonate mixtures may be used, for example tartaric, fumaric, malic or adipic acids and sodium, potassium, calcium (bi)carbonates or sodium glycine carbonate.

The amount of effervescent couple is selected depending on the amount of the standardized or non-standardized extract of Eurycoma longifolia used so as to counter the bitter taste of the extract without itself causing discomfort in the patient's mouth. In certain embodiments, the effervescent couple can comprise about 12, 14, 16, 18, 20, 22, 24, 26, 28, 30, 32, 34, 36, 38, 40, 42, 44, 46, 48, 50, 52, 54 or 55% by weight of the formulation, preferably about 20, 22, 24, 26, 28 or about 30% by weight of the formulation. In at least one embodiment, the effervescent couple comprises about 28% by weight of the formulation. The acidic component of the effervescent couple can comprise about 7, 8, 9, 10, 12, 14, 16, 18, 20, 22, 24, 26, 28 or 30% by weight of the formulation, preferably about 10, 12, 14, 16, 18 or 20% by weight of the formulation. In one embodiment, the acidic component comprises about 12.5% by weight of the formulation. In certain embodiments, the basic component of the effervescent couple can comprise about 5, 7, 9, 11, 13, 15, 17, 19, 21, 23, or 25% by weight of the formulation, preferably about 10, 11, 12, 13, 14 or 15% by weight of the formulation. In at least one embodiment, the basic component comprises about 2.5% by weight of the formulation.

Depending on the amount of the standardized or non-standardized extract of Eurycoma longifolia used in the formulation, the taste masking agents described herein, which generally fall in the class of sweeteners, may not be sufficient to render palatable the bitterness of the standardized extract. Accordingly, a flavoring agent can be included in the formulation to enhance the palatability of the Eurycoma longifolia extract. Flavoring agents can be obtained from either natural or synthetic sources. An illustrative list of such flavorants includes volatile oils, synthetic flavor oils, flavoring-aromatics, oils, liquids, oleoresins and extracts derived from plants, leaves, flowers, fruits, stems and combinations thereof. A non-limiting representative list of these includes citric oils, such as lemon, citron, orange, grape, lime and grapefruit, and fruit essences, including apple, pear, peach, grape, strawberry, raspberry, cherry, plum, pineapple, apricot, or other fruit flavors. Other useful flavorants include aldehydes and esters, such as benzaldehyde (cherry, almond); citral, i.e., alpha-citral (lemon, lime); neral, i.e., beta-citral (lemon, lime); decanal (orange, lemon); aldehyde C-8 (citrus fruits); aldehyde C-9 (citrus fruits); aldehyde C-12 (citrus fruits); tolyl aldehyde (cherry, almond); 2,6-dimethyloctanal (green fruit); 2-dodenal (citrus mandarin); and mixtures thereof.

The amount of flavourant is dependent on the amount of the standardized or non-standardized extract Of Eurycoma longifolia used. Typically, the amount of flavourant comprises about 0.002, 0.004, 0.006, 0.008, 0.01, 0.02, 0.04, 0.06, 0.08, 0.1, 0.2, 0.4, 0.6, 0.8, 1, 1.5, 2, 2.5, 3, 3.5, 4, 4.5, 5, 5.5, 6, 6.5, 7, 7.5, 8, 8.5 or 9% by weight of the formulation. Preferably, the flavourant comprises about 0.1, 0.2, 0.4, 0.6, 0.8, 1, 1.5, 2, 2.5, 3, 3.5 or 4% by weight of the formulation. In at least one embodiment, the preferred flavourant is a combination of citron and menthol. Both racemic and I-menthol can be used, however, I-menthol is preferred. The amount of citron and I-menthol used comprises about 2.5% and 0.2% by weight of the formulation respectively.

The skilled artisan will appreciate that components of the effervescence couple can also serve as flavourants. Accordingly, both the acidic and basic components of the effervescent couple can also function as flavourants. Thus, in at least one embodiment, the herbaceutical formulation comprises about 7.5, 8, 10, 12, 14, 16, 18, 20, 22, 24, 26, 28 or about 30% by weight of the formulation a first flavoring agent selected from the group consisting of anhydrous citric acid, tartaric acid, sodium gluconate, trisodium citrate, fumaric acid, malic acid, adipic acid and mixtures thereof; about 0.1, 0.2, 0.4, 0.6, 0.8, 1, 1.5, 2, 2.5, 3, 3.5, 4, 4.5, 5, 5.5, 6, 6.5, 7, 7.5, 8, 8.5 or 9% by weight of the formulation a second flavoring agent selected from the group consisting of a fruit based flavoring agent; and about 0.002, 0.004, 0.006, 0.008, 0.01, 0.02, 0.04, 0.06, 0.08, 0.1, 0.2 or 0.4% by weight of the formulation a third flavoring agent selected from the group consisting of menthol, mint, thymol and mixtures thereof. In a preferred embodiment, the herbaceutical formulation comprises about 15% by weight of the formulation a first flavoring agent selected from the group consisting of anhydrous citric acid, tartaric acid, sodium gluconate, trisodium citrate, fumaric acid, malic acid, adipic acid and mixtures thereof, about 2.5% by weight of the formulation a second flavoring agent selected from the group consisting of a fruit based flavoring agent, and about 0.2% by weight of the formulation a third flavoring agent selected from the group consisting of menthol, mint, thymol and mixtures thereof.

Other excipients such as lubricants, which can also function as glidants, can be added to the herbaceutical formulation described herein. The role of such excipients is to increase the flowability of the powder by reducing inter-particulate friction. The excipient used for this purpose comprises about 0.05, 0.07, 0.09, 0.1, 0.3, 0.5, 0.7, 0.9, 1, 1.5, 2, 2.5, 3, 3.5, 4, 4.5 or 5% by weight of the formulation. Preferably, the excipient serving this function comprises about 0.1, 0.2, 0:4, 0.6, 0.8, 1, 1.2, 1.4, 1.6, 1.8 or 2% by weight of the formulation. In at least one embodiment, the excipient serving to increase flowability of the powder comprises about 0.5% by weight of the formulation. The lubricant or glidant for use to increase flowability of the powder is selected from the group consisting of magnesium aluminum silicate, hydoxyethyl cellulose, lauric acid, leucine, poloxamers, polyvinyl alcohol, talc, calcium stearate, glycerin monostearate, glyceryl behenate, glyceryl palmitostearate, magnesium lauryl sulfate, sodium lauryl sulfate, sodium stearyl fumarate, stearic acid, magnesium stearate, medium chain triglycerides, palmitic acid, polyethyelene glycol, potassium benzoate, sodium benzoate, zinc stearate and mixtures thereof. In at least one embodiment, the lubricant comprises about 0.5% magnesium aluminum silicate.

Adequate hydration and maintaining proper electrolyte balance is critical during athletic performance. As athletes train and perform they lose fluids and electrolytes via sweat. Hydrating with water alone can help prevent over-heating, but won't protect against electrolyte imbalances that can hinder performance. In some cases drinking only water can dangerously dilute out the electrolytes in the body, actually worsening electrolyte imbalances (hyponatremia). Conversely, consuming an electrolyte containing sports drink can minimize the risk of muscle cramps and fatigue by replacing electrolytes lost in sweat. Well-formulated electrolyte beverages will also enhance fluid absorption and encourage thirst, allowing for more rapid rehydration. Electrolyte replacement beverages have a clear advantage over water for promoting fluid and electrolyte balance, which in turn helps to optimize health and athletic performance.

Accordingly, when the herbaceutical formulation comprising the standardized or non-standardized extract of Eurycoma longifolia described herein is consumed as a sports drink or tonic, the formulation comprises at least one mineral salt. In at least one embodiment, the herbaceutical formulation comprising a standardized or non-standardized extract of Eurycoma longifolia comprises at least one mineral salt selected from the group consisting of sodium chloride, potassium chloride, magnesium chloride, calcium chloride and mixtures thereof. In a preferred embodiment, the formulation comprises a combination of sodium and potassium chloride as the mineral salt. The amount of mineral salt used in the formulation typically comprises about 0.2, 0.4, 0.6, 0.8, 1, 1.2, 1.4, 1.6, 1.8, 2, 2.2, 2.4, 2.6, 2.8, 3, 3.2, 3.4, 3.6, 3.8, 4, 4.2, 4.4, 4.6, 4.8, 5, 5.5, 6, 6.5, 7, 7.5, 8, 8.5, 9, 9.5, 10, 12, 14, 16, 18, 20, 22, 24, 26, 28, or 30% by weight of the formulation. In a preferred embodiment, the amount of sodium chloride and potassium chloride comprises about 0.6% and about 2.5% by weight of the formulation respectively.

The process for manufacturing the formulations described herein are well known to the skilled artisan and can be generally described as follows. The standardized and non-standardized extract of Eurycoma longifolia, were obtained from Biotropics Malaysia Berhad (Shah Alam, Selangor Darul Ehsan, Malaysia), and the Institute of Bioproduct Development, Universiti Teknologi Malaysia, Johor, Malaysia respectively. The non-standardized extract of Hibiscus sabdariffa was obtained from KBioCorp, Kulim, Kedah, Malaysia. The extracts and excipients are preferably in powder form. All components of the formulation are first weighed to the desired quantity per batch size. In order to obtain particles of uniform size, the weighed powder extract and excipients are individually passed through a sieve or sieves of appropriate size. Sieves of different sizes may be used for different excipients.

In the first stage, the sieved extract is first mixed with about 50% of the taste masking agent, flavourant and lubricant/glidant in a stainless steel tumbling mixer. Tumbling mixers are commonly used for mixing/blending of free-flowing powders or granules. The time required to obtain a homogenous mix of the extract, flavourant, and taste masking agent is dependent on the batch size. The bigger the batch size, the longer the time required for obtaining a homogenous mix. Once a homogenous mix is achieved, the remainder of the taste-masking agent and flouvarant are added and again mixed in the tumbler until a homogenous mix is obtained once again. The temperature at which the tumbling is carried out is typically between 22° C.-25° C. Once a homogenous mix is obtained, the mix is sampled for quality assurance testing for the desired quality and taste characteristics. In the second stage, one component of the effervescence couple together with the remainder of the lubricant/glidant is added and again mixed for a specified period of time in the same tumbler under the same conditions. At the final stage of mixing, the second component of the effervescent couple is added to complete mixing of the herbaceutical formulation of Eurycoma longifolia. The order in which the excipients are added to the extract is not critical. The at least one mineral salt can be added at any stage.

The final formulation is then loaded into the hopper of a sachet-filling machine, and the sachets are filled and sealed to the desired weight with the formulation. The sachets are sent for quality assurance testing to ensure that the filling and sealing of the sachets meet the required parameters. The sachets are then sent for final packing into secondary box packing and subsequent shipment.

It should be appreciated that the formulation does not have to manufactured with all of the components and packaged into a single sachet. To avoid premature activation of the effervescence couple due to high humidity, it may be advantageous to manufacture the formulation as a divided powder. For example, the formulation can be manufactured in the absence of one component of the effervescence couple. The formulation can then be packaged into two separate sachets, one comprising the formulation without one of the components of the effervescence couple and the other with the second component of the effervescence couple that was left out of the formulation. The patient can reconstitute the formulation by emptying both sachets into an aqueous beverage of choice.

Packaged Compositions

Packaged effervescent herbaceutical formulations are included herein. Such packaged formulations include one or more formulations described herein comprising standardized, non-standardized or combinations of standardized or non-standardized herbaceutical extracts. The formulations described herein can be provided as unit dosage forms in sachets and instructions for using the dosage form to treat a patient having a disease or disorder responsive to the actives in the standardized or non-standardized herbaceutical extracts.

The packaged formulations include providing prescribing information, over the counter medical use information, or nutritional information for the dosage foim, for example, to a patient or health care provider, or as a label in a packaged pharmaceutical formulation. Information included on the pharmaceutical package may include for example efficacy, dosage and administration, contraindication and adverse reaction information pertaining to a standardized or non-standardized herbaceutical extract. The dosage and administration information, for example, can include, but is not limited to dosing frequency.

In certain embodiments the effervescent herbaceutical formulations provided herein are in the form of a single powder or a divided powder provided in one sachet or in separate sachets together with over the counter medical use information and/or nutritional information.

The packaged herbaceutical formulations can comprise one ore more of the standardized or non-standardized herbaceutical extracts described herein as the only active agent. In other embodiments, one or more of the standardized or non-standardized compositions described herein can be packaged in combination with one or more other active agents or dietary supplements such as for example, flavonoids, anti-oxidants, nutritional supplements and mixtures thereof. Examples of nutritional supplements include, but are not limited to glucosamine, chondroitin sulfate, methylsulfonylmethane, bromelain and mixtures thereof.

The invention is illustrated by the following examples.

### Example 1

The following table provides the ingredients and their respective amounts for formulating an herbaceutical effervescent formulation comprising a standardized extract of Eurycoma longifolia according to one embodiment:

| | | AMOUNT | AMOUNT |
|---|---|---|---|
| FUNCTION | INGREDIENTS | (WT/WT) | (GM) |
| Active Extract | Standardized extract of | 1.1% | 0.1 |
| | Eurycoma longifolia | | |
| Taste Masking | Glucose | 66.28% | 6 |
| Agent | Sucralose | 1.67% | 0.15 |
| | β-Cyclodextrin | 1.1% | 0.1 |
| Effervescent | Anhydrous Citric Acid | 13.25% | 1.2 |
| Couple | Anhydrous Sodium | 11.0% | 1 |
| | Bicarbonate | | |
| Flavourant | Citron | 2.23% | 0.2 |
| | I-Menthol | 0.14% | 0.013 |
| Lubricant/Glidant | Neusilin | 0.44% | 0.04 |
| | Grade UFL2 | | |
| Mineral Salt | Potassium Chloride | 2.24% | 0.2 |
| | Sodium Chloride | 0.55% | 0.05 |
| Total | | 100% | 9.053 |

All ingredients, in powder form, comprising the formulation are first weighed according to the proportions of each ingredient for the desired batch size. The extract, glucose, sucralose, effervescent couple, lubricant/glidant and mineral salt are then passed through a 250μ (#60) sieve. The β-cyclodextrin is passed through a 420μ sieve.

In Stage I of the formulation process, the weighed and sieved extract is placed in a stainless steel automatic tumbler mixer (Kimah Industrial Supplier, Penang, Malaysia) together with all of the sucralose, I-menthol and β-cyclodextrin for a few minutes. This is followed with the addition of glucose and citron, and mixed for a few more minutes until a homogenous mixture is achieved. At the end of Stage I, which is about 10-15 minutes long, homogenous mixture is sampled for flow characteristics. The parameters tested were bulk density (0.833), tapped density (0.961), angle of repose (0.488), Hausner ratio (1.153) and Carr's Index (13.32). Methods used for measuring these parameters are well known in the art.

In Stage II, the formulation manufacture is continued with the addition of all of the anhydrous citric acid, mineral salt and 50% of the lubricant/glidant. The powder is mixed for a few minutes before the remaining lubricant/glidant is added. The powder at this stage is mixed for about 10-15 minutes.

The anhydrous sodium bicarbonate is added at Stage II and mixed for an additional 10-15 minutes. At the end of Stage III, the formulation is sampled and sent for quality assurance testing. The flow characteristics of the final formulation are again tested for the bulk density and angle of repose using the same methods at the end of Stage I.

The entire mixing process is carried out at about 22° C.-25° C.

Once the final formulation satisfies the quality assurance tests, the formulation is loaded in to the hopper of a sachet-filling machine (Model DXD.K-150, Kimah Automatic Granule Packaging Machine, Penang, Malaysia). Each sachet is filled to about 9 gm. The filled sachets are then sent for final packaging in secondary box packaging.

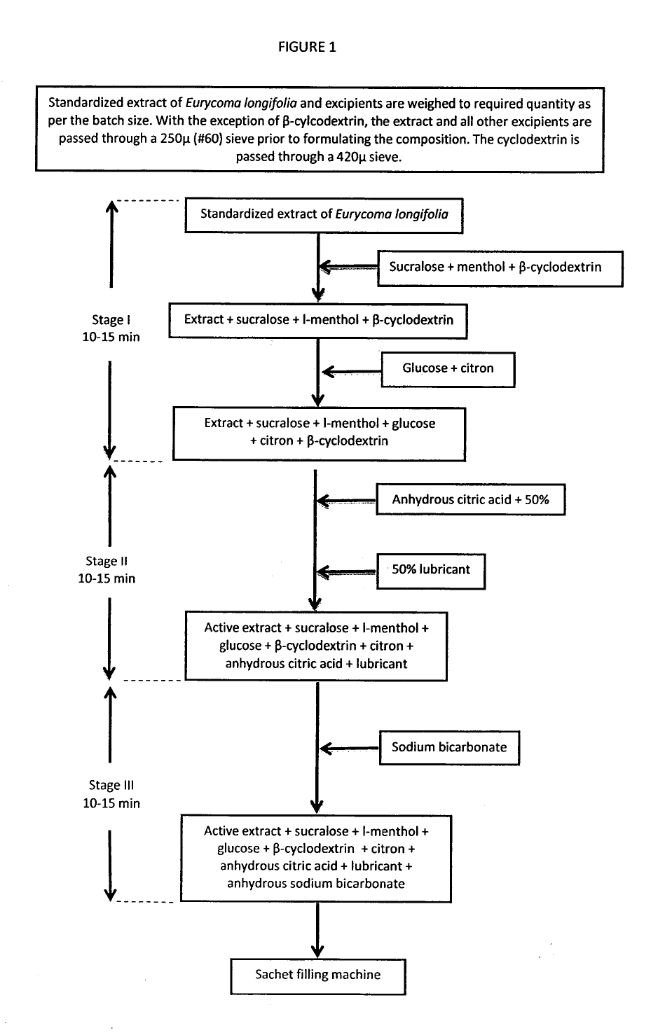

FIG. 1 is illustrative of the manufacturing process of the formulation.

### Example 2

The following table provides the ingredients and their respective amounts for formulating an herbaceutical effervescent formulation comprising a non-standardized extract of Eurycoma longifolia in combination with a non-standardized extract of Hibiscus sabdariffa according to one embodiment:

| | | AMOUNT | AMOUNT |
|---|---|---|---|
| FUNCTION | INGREDIENTS | (WT/WT) | (GM) |
| Active Extract | Standardized extract of | 1.05% | 0.1 |
| | Eurycoma longifolia | | |
| Taste Masking | Glucose | 62.8% | 6 |
| Agent | Sucralose | 1.57% | 0.15 |
| | β-Cyclodextrin | 1.05% | 0.1 |
| Effervescent | Anhydrous Citric Acid | 12.56% | 1.2 |
| Couple | Anhydrous Sodium | 10.47% | 1 |
| | Bicarbonate | | |
| Flavourant | Citron | 2.09% | 0.2 |
| | I-Menthol | 0.14% | 0.013 |
| | Non-Standardized extract | 5.23% | 0.5 |
| | of Hibiscus sabdariffa | | |
| Lubricant/Glidant | Neusilin | 7.23% | 0.04 |
| | Grade UFL2 | | |
| Mineral Salt | Potassium Chloride | 2.09% | 0.2 |
| | Sodium Chloride | 0.52% | 0.05 |
| Total | | 100% | 9.55 |

The method of manufacture of the above formulation is as described for the formulation in Example 1.

1-39. (canceled)

40. A herbaceutical formulation comprising:
a. An effective amount of a non-standardized extract of Eurycoma longifolia;
b. about 0.05% to about 90% by weight of said formulation at least one taste masking agent;
c. about 5% to about 30% by weight of said formulation at least one effervescent couple;
d. about 0.5% to about 30% by weight of said formulation at least one flavoring agent;
e. about 0.05% to about 5% by weight of said formulation at least one lubricant; and,
f. about 0.2% to about 5% by weight of said formulation at least one electrolyte.

41. The formulation of claim 40, wherein the at least one taste masking agent is selected from the group consisting of monosaccharides, disaccharides, sugar alcohols, polydextrose, dextrates, dextrin, dextrose anhydrous, sugar substitutes and mixtures thereof.

42. The formulation of claim 40, wherein the effervescent couple comprises at least one acidic component comprising about 7.5% to about 30% by weight of said formulation and wherein the acidic component is selected from the group consisting of anhydrous citric acid, tartaric acid, sodium gluconate, trisodium citrate, fumaric acid, malic acid, adipic acid or any acid salts thereof; and at least one alkaline component comprising about 5% to about 25% by weight of said formulation wherein the alkaline component is selected from the group consisting of anhydrous sodium, potassium, calcium (bi) carbonates, sodium glycine carbonates and mixtures thereof.

43. The formulation of claim 40 wherein the at least one flavoring agent is selected from the group consisting of citron, menthol, mint, thymol, a flavoring agent derived from fruit, anhydrous citric acid, tartaric acid, malic acid, sodium gluconate, trisodium citrate, fumaric acid, adipic acid and mixtures thereof.

44. The formulation of claim 40 wherein the at least one lubricant is selected from the group consisting of magnesium aluminum silicate, hydroxyethyl cellulose, lauric acid, leucine, poloxamers, polyvinyl alcohol, talc, calcium stearate, glyceryl monostearate, glyceryl behenate, glyceryl palmitostearate, magnesium lauryl sulfate, sodium lauryl sulfate, sodium stearyl fumarate, stearic acid, magnesium stearate, medium chain triglycerides, palmitic acid, polyethylene glycol, potassium benzoate, sodium benzoate, zinc stearate and mixtures thereof.

45. The formulation of claim 40 wherein the at least one electrolyte comprises about 0.8% to about 5% potassium chloride and about 0.2% to about 1.25% sodium chloride by weight of said formulation.

46. The formulation of claim 40 in combination with an effective amount of a non-standardized extract selected from the group of non-standardized extracts of Orthosiphon stamineus, Labisia pumila, Andrographis paniculata, Phyllanthus niruri, Hibiscus sabdariffa and mixtures thereof.

47. The formulation of claim 40 in combination with an effective amount of a compound selected from the group consisting of flavonoids, anti-oxidants, nutritional supplements and mixtures thereof.

48. The formulation of claim 40 wherein the nutritional supplement is selected from the group consisting of glucosamine, chondroitin sulfate, methyisnifonylmethane, bromelain and mixtures thereof.

49. A method of enhancing athletic performance in a human subject comprising administering the formulation of claim 40 as a sports drink to said human subject.

50. A method of enhancing sexual libido in a human subject comprising administering the formulation of claim 40 to said human subject in need of such administration.

51. The herbaceutical formulation of claim 40 in a package together with instructions for using said formulation to enhance athletic performance.

52. The herbaceutical formulation of claim 46 in a package together with instructions for using said formulation to enhance athletic performance.

53. The herbaceutical formulation of claim 40 in a package together with instructions for using said formulation to increase sexual libido in a human subject.

54. The herbaceutical formulation of claim 46 in a package together with instructions for using said formulation to increase sexual libido.

55. The herbaceutical formulation comprising of claim 40 in a package together with instructions for using said formulation to treat male infertility, male sexual dysfunction, increasing testosterone synthesis, increasing testosterone release from testis cells, increasing sperm count, increasing sperm motility, treating the symptoms of late-onset hypoganadism or the management of hypogonadism in a human male in need of such treatment.

56. The herbaceutical formulation of claim 46 in a package together with instructions for using said formulation to treat male infertility, male sexual dysfunction, increasing testosterone synthesis, increasing testosterone release from testis cells, increasing sperm count, increasing sperm motility, treating the symptoms of late-onset hypoganadism or the management of hypogonadism in a human male in need of such treatment.
